(19) United States
(12) Patent Application Publication
(10) Pub. No.: US 2019/0261912 A1
(43) Pub. Date: Aug. 29, 2019

(54) SYSTEM AND METHODS FOR IDENTIFYING OR ASSESSING INFLAMMATORY BOWEL DISEASE OR GUT INFLAMMATION

(71) Applicant: THE BIONICS INSTITUTE OF AUSTRALIA, East Melbourne, Victoria (AU)

(72) Inventors: Robert K. Shepherd, East Melbourne, Victoria (AU); John Furness, East Melbourne, Victoria (AU); James Fallon, East Melbourne, Victoria (JP); Sophie Payne, East Melbourne, Victoria (AU); Owen Burns, East Melbourne, Victoria (AU)

(21) Appl. No.: 16/335,907

(22) PCT Filed: Sep. 29, 2017
(86) PCT No.: PCT/AU2017/051072

§ 371 Date: Mar. 22, 2019

(30) Foreign Application Priority Data
Sep. 29, 2016 (AU) 2016903957

(51) Int. Cl. A61B 5/00; A61B 5/04; A61B 5/053

(52) U.S. Cl. CPC A61B 5/4255; A61B 5/6852; A61B 5/0538; A61B 5/04

(57) ABSTRACT

Detecting of a disorder associated with impaired barrier function in the gastrointestinal tract of a subject is described, including through determining bioimpedance across a subject's gastrointestinal tract wall and detecting impaired barrier function based on bioimpedance. An electrical signal is measured between first and second electrodes, at least the first electrode being located in a gastrointestinal tract of the body and one or more characteristics are determined based on the measured electrical signal. The one or more characteristics can comprise hallmarks of impaired barrier function such as leakiness and/or permeability of the gastrointestinal tract and can provide an indication of inflammatory bowel disease and/or gut inflammation.

### TECHNICAL FIELD

The present invention relates to a system and methods for identifying or assessing inflammatory bowel disease or gut inflammation.

### BACKGROUND

Inflammatory bowel disease (IBD) is a debilitating, relapsing condition that first emerges in young adulthood and can affect a patient throughout their life. IBD covers a group of disorders in which the gastrointestinal tract becomes inflamed. Major types of IBD include Crohn's disease, in which inflammation affects the full thickness of the bowel wall anywhere along the gastrointestinal tract, and ulcerative colitis, in which inflammation affects the inner lining (mucosa) of the colon and rectum.

Current clinical methods of monitoring IBD involve endoscopy, biopsy, blood tests and faecal tests. These methods, however, suffer from a number of drawbacks. Endoscopy is a procedure that involves the subjective visual inspection of the gastrointestinal tract using a flexible endoscope, which is fed down the esophagus into the stomach and the small intestines of a patient, or fed intra-rectally. A small camera on the endoscope transmits an image to a monitor, allowing the clinician to closely examine the intestinal lining. Endoscopy provides qualitative results in real-time. On the other hand, biopsy and blood/faecal tests provide quantitative, objective measures to monitoring IBD, but not in real-time. Such methods can be invasive, inconvenient and require considerable time before test results are available. These existing clinical methods therefore do not provide real-time objective measurement of IBD.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Visual inspection for gross morphological changes and histology are often relied on to assess barrier function (e.g. leakiness and/or permeability of gastrointestinal tract). Although, the gastrointestinal tract is a complex, multilayer system, the present inventors have found that changes in permeability and/or leakiness of the gut wall following inflammation is in direct relation with changes in bioimpedance across the gut wall. These findings suggest that bioimpedance can be used to detect impaired barrier function in the gastrointestinal tract. Furthermore, with impaired barrier function being associated with various inflammatory disorders such as inflammatory bowel disease, the findings of the inventors suggest that bioimpedance can be used to detect inflammatory disorders of this nature. According to an aspect of the present disclosure, there is provided a method of identifying or assessing inflammatory bowel disease, the method comprising:

measuring an electrical signal between first and second electrodes, the first and second electrodes being located relative to a body, at least the first electrode being located in a gastrointestinal tract of the body; and determining one or more characteristics of inflammatory bowel disease based on the measured electrical signal.

In some embodiments, the one or more characteristics may comprise hallmarks of impaired barrier function such as leakiness and/or permeability of the gastrointestinal tract. The one or more characteristics may also comprise inflammation of a wall of the gastrointestinal tract.

Accordingly, in another aspect, the methods of the present disclosure encompass identifying or assessing impaired barrier function in the gastrointestinal tract, the method comprising measuring an electrical signal between first and second electrodes, the first and second electrodes being located relative to a body, at least the first electrode being located in a gastrointestinal tract of the body; and determining reduced barrier function based on the measured electrical signal.

In another aspect of the present disclosure provides a method of identifying or assessing gut inflammation comprising:

measuring an electrical signal between first and second electrodes, the first and second electrodes being located relative to a body, at least the first electrode being located in a gastrointestinal tract of the body; and determining an inflammation of a wall of the gastrointestinal tract based on the measured electrical signal.

In another aspect, the methods of the present disclosure encompass detecting a disorder associated with impaired barrier function in the gastrointestinal tract of a subject, the method comprising determining bioimpedance across a subject's gastrointestinal tract wall and detecting impaired barrier function based on bioimpedance. In this aspect, impaired barrier function may be detected based on a decrease in bioimpedance relative to a control.

In another aspect, the methods of the present disclosure encompass detecting an inflammatory disorder in a human subject, the method comprising determining bioimpedance across a subject's gastrointestinal tract wall and detecting a decrease in bioimpedance relative to a control. In one example, the gastrointestinal tract wall may be a wall of the esophagus. In another example, the gastrointestinal tract wall may include a columnar epithelium. In another example, the gastrointestinal tract wall may be a wall of the duodenum. In another example, the gastrointestinal tract wall may be a wall of the colon (ascending transverse or descending). In another example, the gastrointestinal tract wall may be a wall of the rectum. In another example, the gastrointestinal tract wall may be a wall of the stomach. In another example, the gastrointestinal tract wall may be a wall of the jejunum. In another example, the gastrointestinal tract wall may be a wall of the Ileum.

In another aspect, the methods of the present disclosure encompass resolving an inconclusive clinical assessment for inflammatory bowel disease, the method comprising determining bioimpedance across a subject's gut wall, wherein a decrease in bioimpedance relative to a control indicates the presence of inflammatory bowel disease.

In the above aspects, various inflammatory disorders may be detected or assessed. In an example, the inflammatory disorder is inflammatory bowel disease. In other examples, the inflammatory disorder is selected from the group consisting of Crohn's disease, ulcerative colitis, coeliac disease, protein losing enteropathy, non-alcoholic fatty liver disease and non-invasive reflux disease (NERD), gastritis or leaky gut.

In any of the above aspects and embodiments, the second electrode may be located externally of the gastrointestinal tract. For example, the second electrode may be subcutaneous.

The measured electrical signal may be indicative of bioimpedance of tissue between the first and second electrodes.

The above methods may comprise determining bioimpedance of tissue between the first and second electrodes based on the measured electrical signal, and may also comprise determining the one or more characteristics based on the determined bioimpedance.

An electrical current may be applied across tissue between the first and second electrodes and the electrical signal may be measured in response to the applied electrical current. The electrical current may be applied via the first and second electrodes and/or applied via one or more additional electrodes.

In an example, bioimpedance is determined by measuring an electrical signal between intra-luminal and extra-luminal electrodes. In an example, the measured signal is current. In another example, the measured signal is voltage. For example, the measured voltage can be the peak voltage transient.

In another example, the measured signal is based on a biphasic electrical pulse delivered between the intra-luminal and extra-luminal electrodes. In an example, the biphasic pulse has a pulse width of between 20 μs and 200 μs per phase. For example, the biphasic pulse can have a pulse width of about 25 μs per phase.

In an example, a reduction in bioimpedance of about 5% relative to a control indicates impaired barrier function in the gastrointestinal tract of a subject and/or an inflammatory disorder. In another example, a reduction in bioimpedance of about 10% relative to a control indicates impaired barrier function in the gastrointestinal tract of a subject and/or an inflammatory disorder.

In an example, intra and extra-luminal electrodes are positioned in the subject's gastrointestinal tract wall. For example, the intra and extra-luminal electrodes can be positioned in the subject's gut wall.

In another aspect, the methods of the present disclosure further comprise treating impaired barrier function and/or an inflammatory disorder by electrically stimulating the Vagus Nerve (VN). For example, VN stimulation can be applied to a subject when a reduction in impedance is detected using an above referenced method. For example, VN stimulation can be applied when a reduction in impedance is detected across a subject relative to control. Accordingly, in an example, the present disclosure encompasses a method of treating impaired barrier function, the method comprising stimulating a subject's vagus nerve when a decrease in bioimpedance is detected across the subject's gastrointestinal tract wall.

In yet another aspect of the present disclosure, there is provided a system for identifying or assessing inflammatory bowel disease or gut inflammation, the system comprising:

a device insertable or implantable into a gastrointestinal tract of a body, the device having at least one first electrode;

a second electrode; and

a processing apparatus configured to determine one or more characteristics of inflammatory bowel disease or gut inflammation based on an electrical signal measured between the at least one first electrode and the second electrode.

The one or more characteristics may comprise inflammation of the wall of the gastrointestinal tract.

In some embodiments, the second electrode may be configured to locate externally of the gastrointestinal tract. In other embodiments, the second electrode may be configured to locate in the peritoneal cavity of the body. In alternative embodiments, the second electrode may be configured to locate at a skin surface of the body. The second electrode may be positioned on the skin surface, e.g. as part of an electrode patch, or the second electrode may be inserted subcutaneously, e.g. as part of a needle.

The measured electrical signal may be indicative of bioimpedance of tissue between the at least one first electrode and the second electrode.

The processing apparatus may be configured to determine bioimpedance of tissue between the at least one first electrode and the second electrode based on the measured electrical signal. The processing apparatus may be configured to determine the one or more characteristics based on the determined bioimpedance. The processing apparatus may be configured to apply electrical current across tissue between the at least one first electrode and the second electrode and the measured electrical signal may be in response to the applied electrical current.

The electrical current may be applied via the at least one first electrode and the second electrode and/or applied via one or more additional electrodes.

The processing apparatus may comprise a stimulator, which is configured to apply the electrical current.

The at least one first electrode may comprise a plurality of first electrodes and the processing apparatus may be configured to determine one or more characteristics of inflammatory bowel disease or gut inflammation based on a plurality of electrical signals measured between the second electrode and different first electrodes.

The insertable device may comprise an endoscope, and the endoscope may have a flexible body with a proximal end and a distal end. The endoscope may comprise any one or more of a channel for the supply of gas and/or liquid into the gastrointestinal tract, an instrument channel, an optical channel, a light channel and an electrode channel.

The optical channel may comprise a camera module for transmitting images from the distal end of the endoscope to the processing apparatus, the light channel may comprise a light source for transmitting light to the distal end of the endoscope, and the at least one first electrode may be provided in the electrode channel at or adjacent the distal end of the endoscope.

The implantable device may comprise a flexible lead body having a distal portion, a proximal portion and a transition portion connecting the distal portion and the proximal portion. The distal portion may be configured to be implanted in the gastrointestinal tract, and the proximal portion may be configured to be located external of the gastrointestinal tract.

The transition portion may comprise a central portion and first and second swelled portions on either side of the central portion. The first and second swelled portions may connect the proximal and distal portions, respectively. The central portion may have a maximum diameter less than a maximum diameter of the first and second swelled portions, and the first and second swelled portions may be configured to prevent migration of the device from an implantation site when positioned for use.

The lead body may be formed of silicone or other flexible material and may be of a generally Z-shaped configuration.

The at least one first electrode may be provided at the distal portion of the lead body, and the second electrode may be provided at the proximal portion of the lead body. The first and second electrodes may be band electrodes. Electrodes used herein may be formed of any suitable electrically conductive and biocompatible material, such as platinum, platinum iridium alloy or otherwise.

The above system may also comprise a lead wire extending through the lead body for coupling the first and second electrodes to the processing apparatus. The lead wire may be formed of any suitable electrically conductive and biocompatible material such as platinum, platinum iridium alloy or otherwise. In an example, the lead wire is inserted through a body orifice.

In a further aspect of the present disclosure, there is provided a method comprising:

inserting or implanting a device into a gastrointestinal tract of a body, the device comprising at least one first electrode;

locating a second electrode relative to the body;

coupling a processing apparatus to the at least one first electrode and the second electrode, the processing apparatus being configured to determine an inflammation of a wall of the gastrointestinal tract based on an electrical signal measured between the at least one electrode and the second electrode.

In some embodiments, the second electrode may be located externally of the gastrointestinal tract. In other embodiments, the second electrode may be located in the peritoneal cavity of the body. In alternative embodiments, the second electrode may be located on a skin surface of the body.

Inserting or implanting the device may comprise anchoring the device to the wall of the gastrointestinal tract at or adjacent a region of gut wall inflammation.

### BRIEF DESCRIPTION OF DRAWINGS

Embodiments of the invention will now be described, by way of example only, with reference to the accompanying drawings, in which:

FIG. 1. Schematic view of a system according to an embodiment of the present disclosure.

FIG. 2a. Perspective view of a device used in the system of FIG. 1.

FIG. 2b. Enlarged perspective view of ‘A’ of FIG. 2a.

FIG. 3. Enlarged end view of the device of FIG. 2a.

FIG. 4. Schematic illustration of use of the system of FIG. 1.

FIG. 5a. Isometric view of a device used in a system according to another embodiment of the present disclosure.

FIG. 5b. Enlarged isometric view of 13′ of FIG. 5a.

FIG. 5c. Front view of the device of FIG. 5a.

FIG. 6a. Schematic illustration of an experimental model used to identify induced gut inflammation.

FIG. 6b. Graph showing changes in gut impedance following inflammation, obtained from the experimental model of FIG. 6a over a period of 3 hours.

FIG. 7-1. Schematic diagram of gut and hypothetical circuit diagram. A-B: Schematic diagram of control (A) gut show that the epithelial cell layer (Ai) adhered together by tight junctions form a high impedance barrier between intra-luminal and extra-luminal space. Within inflammed gut tissue (B), epithelial cells are lost and/or tight junctions disrupted (Bi), thereby allowing charge to flow through these pathways. C-D: Schematic examples of voltage transients induced across control (C) and inflamed (D) ileum, in response to a 25 us biphasic current pulse. The peak voltage transient (Vₜₒₜₐₗ) is measured at the end of the first phase. E: Schematic circuit diagram depicts that the in vivo voltage measured is effected by the intraluminal electrode, intraluminal content, epithelium, body tissue and fluid and subcutaneous electrode. During inflammation, the epithelium resistive component is reduced resulting in a decrease in the peak voltage transient. Exemplary stimulation paramaters are provided in FIG. 7-2.

FIG. 8. Inflammation-induced changes in ileum tissue following intraluminal TNBS injection. A, C: Images show comparison of the villi. In A, the surface epithelium was intact at the tips of the villi (arrows). Lymphatic vessels (asterisks) were apparent but there were no red blood cells in the connective tissue spaces. In C, the villi were shortened and the base of the mucosa can be seen in the same field as villus tips. The surface epithelium was lost from the tips of the villi (arrows). Numerous regions of microhemorrhage were seen in the villi (m). B, D: Images show comparison of the crypt region. After TNBS injection, there was marked accumulation of neutrophils in the venules (ven; indicated by arrows in D), which was not seen in control tissue (C). The crypts were intact in both control and TNBS treated tissue. Eosinophils (circled) were more numerous in inflamed tissue (D), than in control (B). E: Histologically scored tissue damage was significantly more prevalent within inflamed tissue (n=18), than in control (E1; n=6). Data shows mean (±S.E.M), and differences were considered significant for P<0.05. Scale bars in A-B represent 50 μm; Ai-Bi: 20 μm.

FIG. 9. Infiltration of eosinophils into the mucosa correlates with the decrease in gut wall impedance following TNBS injection. A-B: Eosinophils (indicated with arrows) were seen in control mucosa layers (A), but were substantially more prevalent in inflamed mucosa tissue (B). C: Significant infiltration of eosinophils into the mucosa was seen in inflamed tissue (E2-E4; n=18) compared to control (E1; n=6). D: Eosinophil infiltration in the mucosa and normalised gut wall impedance at 3 hours post TNBS injection is significantly correlated (R=−0.66). Data in C show mean±S.E.M, and in D mean eosinophil density data that correlates with normalised gut wall impedance was generated from that same area of tissue. Differences were considered significant for P<0.05. Scale bar for A-B: 50 μm.

FIG. 10. Infiltration of neutrophils into the mucosa correlates with decreases in gut wall impedance following TNBS injection. A-B: Sections from control (A) and inflamed tissue (B) were counterstained with methyl green and neutrophils were identified by their myeloperoxidase activity (MPO, indicated by arrows). C: Following TNBS injection, the number of MPO+ cells doubled within the inflamed mucosa at all electrode locations (E2-E4; n=18). D: There was a significant correlation (R²=−0.65) between normalised gut wall impedance and MPO density, across all electrode locations. Data in C show mean±S.E.M. and in D mean MPO density data that correlates with gut wall impedance generated from that same area of tissue. Differences were considered significant for P<0.05. Scale bar for A-B: 50 μm.

FIG. 11. Infiltration of T cells into the mucosa correlates with decreases in gut wall impedance following TNBS injection. A-B: CD3+ cells were identified in Hematoxylin counter stained sections (indicated by arrows) in control (A) and inflamed (B) tissue. C: Following TNBS injection, the number of CD3+ cells doubled within inflamed mucosa tissue from all electrode locations (E2-E4; n=18). D: There was a significant correlation (R²=−0.57) between normalised gut wall impedance and CD3+ cell density, across all electrode locations. Data in C show mean±S.E.M, and in D mean CD3+ cell density data that correlates with gut wall impedance. Differences were considered significant for P<0.05. Scale bar=50 μm.

FIG. 12. Dose dependant comparison of impedance. Impedance measurements were generated using biomarker array implanted into the ileum. Impedances of awake rats were generated following saline or TNBS injection. C: 0.1% TNBS injection. D: 1% TNBS injection.

FIG. 13. Acute inflammatory changes in sheep ileum following TNBS injection. A-C: Macroscopic view shows vasodilation (B) and significant inflammatory-associated damage to villi compared to control (A, Ai, C). D: Significant decrease in transmucosal impedance in inflamed region.

FIG. 14. Front view of a device used in a system according to another embodiment of the present disclosure.

FIG. 15a. Enlarged front view of ‘C’ of FIG. 14.

FIG. 15b. Enlarged front view of ‘D’ of FIG. 14.

FIG. 15c. Enlarged end view of ‘E’ of FIG. 14.

FIG. 15d. Enlarged top view of ‘E’ of FIG. 14.

FIG. 15e. Enlarged front view of ‘E’ of FIG. 14.

FIG. 16-1. VNS alleviated histological damage in gut tissue. A: Normal ileum (A, Ai) had undamaged, intact surface epithelium (arrows, Ai). B, Bi: At 4 hours following TNBS injection (the time that active VN stimulation was applied) extensive epithelial cell loss (arrow, B) and leukocyte infiltration (circle) was observed. FIG. 16-2 A, Ai: At 5 days following TNBS injection, there was extensive damage to villi (Ai, arrows), leukocyte infiltration in venules (circle) and micro-hemorrhages (m) in unstimulated tissue. Villi architecture were severely disrupted. B, Bi: In VN stimulated tissue, signs of histological damage was reduced. Villi were long and undamaged. Surface epithelium were intact (arrows) although some microhemorrhages were observed (m). Scale bars in A-D: 100 μm; in Ai-Di: 20 μm. FIG. 16-3: E: Histologically scored damage. F-H: At 5 days following TNBS injection, the prevalence of neutrophils increased (P<0.05) within mucosa (F), submucosa (G) and muscle layers (H), but decreased in VNS treated tissue (P<0.05).

### DESCRIPTION OF EMBODIMENTS

The present disclosure relates to a system and methods of identifying or assessing inflammatory bowel disease (IBD) or gut inflammation. During IBD, the wall of the gastrointestinal tract becomes inflamed and as a consequence the permeability and/or leakiness of the wall of the gastrointestinal tract increases. These are key quantitative characteristics of IBD. The present disclosure recognises that changes in permeability and/or leakiness of the gut wall following inflammation is in direct relation with changes in bioimpedance across the gut wall and bioimpedance monitoring can therefore be used to monitor for inflammatory bowel disease (IBD) or gut inflammation.

In the present disclosure, the term “bioimpedance” is intended to refer to the resistance of the body or components of the body such as the gastrointestinal tract to the passage of an electrical signal. In an example, the electrical signal is current. In another example, the electrical signal is voltage. Accordingly, bioimpedance may be measured, for example, by detecting the voltage difference between two electrodes in response to a controlled electrical current that passes between the electrodes, or by detecting the electrical current that passes between the electrodes in response to a controlled voltage difference between the electrodes, or by any combination thereof. In another example, the electrical signal is based on a biphasic electrical pulse delivered between the intra-luminal and extra-luminal electrodes. However, various other examples are discussed below.

Some embodiments of the present disclosure provide a device insertable or implantable in the gastrointestinal tract of a body, the device having at least one electrode that measures bioimpedance across the wall of the gastrointestinal tract. The device may be inserted temporarily into the gastrointestinal tract in association with endoscopy procedures or implanted permanently or semi-permanently into the gastrointestinal tract for continuous monitoring over a period of time.

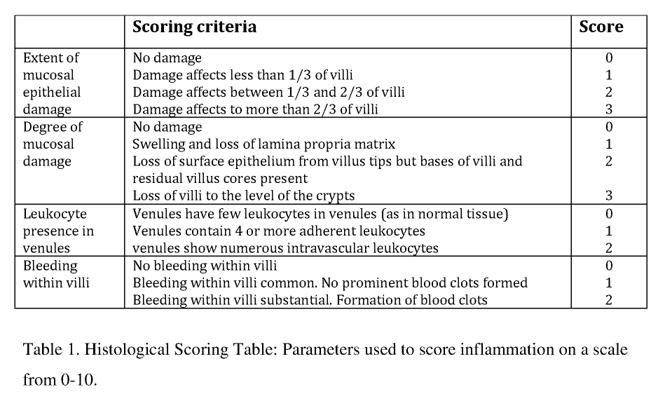

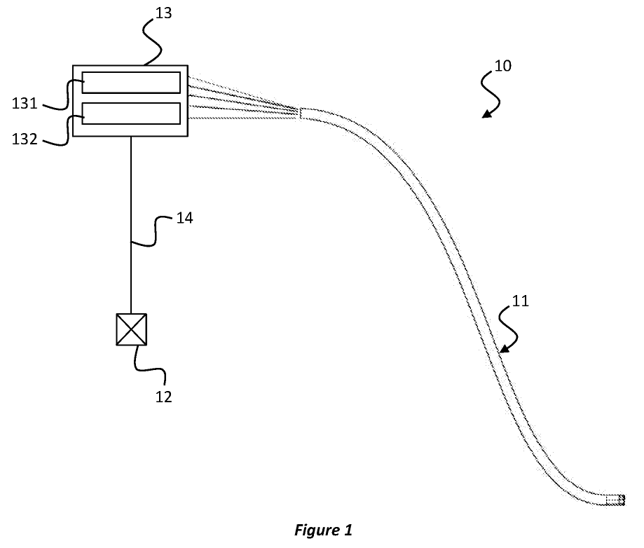

FIG. 1 shows a general view of a system 10 according to an embodiment of the present disclosure. The system 10 may be employed for temporary real-time evaluation of IBD or gut inflammation. The system 10 comprises a device 11 configured to be inserted into a gastrointestinal tract of a body, the device 11 having at least one first electrode 201 (see FIG. 3). The system 10 also comprises a second (reference) electrode 12 and a processing apparatus 13 coupled to the device 11, including the reference electrode 12, via one or more leads 14. The reference electrode 12 may be an electrocardiogram (ECG) patch electrode adapted for placement on the skin surface of the body, for example.

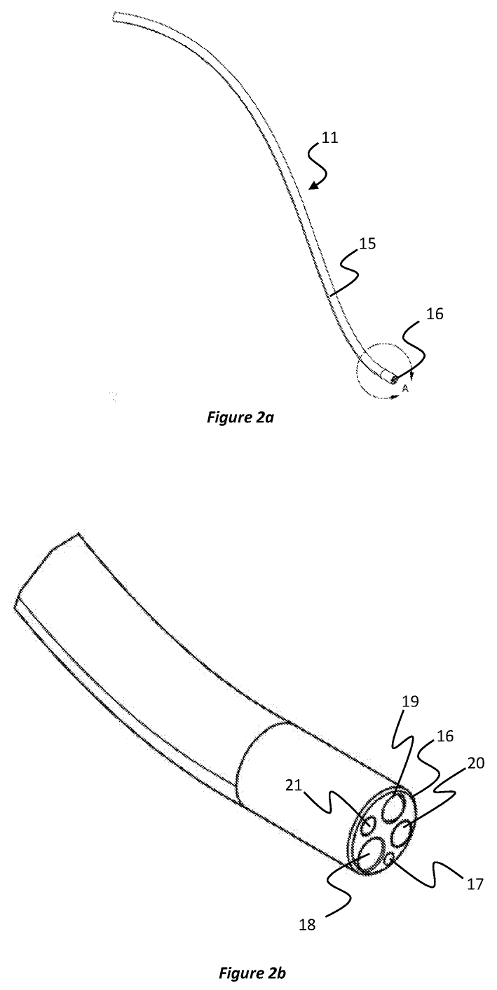

As best shown in FIGS. 2a and 2b, the device 11 is an endoscope adapted for insertion into the gastrointestinal tract. The endoscope 11 has a flexible body 15 with a proximal end (not shown) and distal end 16. With reference to FIGS. 2b and 3, the endoscope 11 can include any one or more of a channel 17 for the supply of gas and/or liquid into the gastrointestinal tract, an instrument channel 18, an optical channel 19, a light channel 20 and an electrode channel 21. The optical channel 19 may comprise a camera module for transmitting images from the distal end 16 of the endoscope 11 to the processing apparatus 13. The light channel 20 comprises a light source for transmitting light to the distal end 16 of the endoscope 11. The at least one first electrode 201 is provided in the electrode channel 21 at or adjacent the distal end 16 of the endoscope 11. In some embodiments, the electrode channel 21 comprises a plurality of first electrodes 201. The at least one first electrode may be formed of any suitable electrically conductive and biocompatible material, such as a platinum iridium alloy or otherwise.

Referring again to FIG. 1, the processing apparatus 13 comprises a stimulator 131 for generating an electrical current and a processor 132 for receiving and processing data from the endoscope 11.

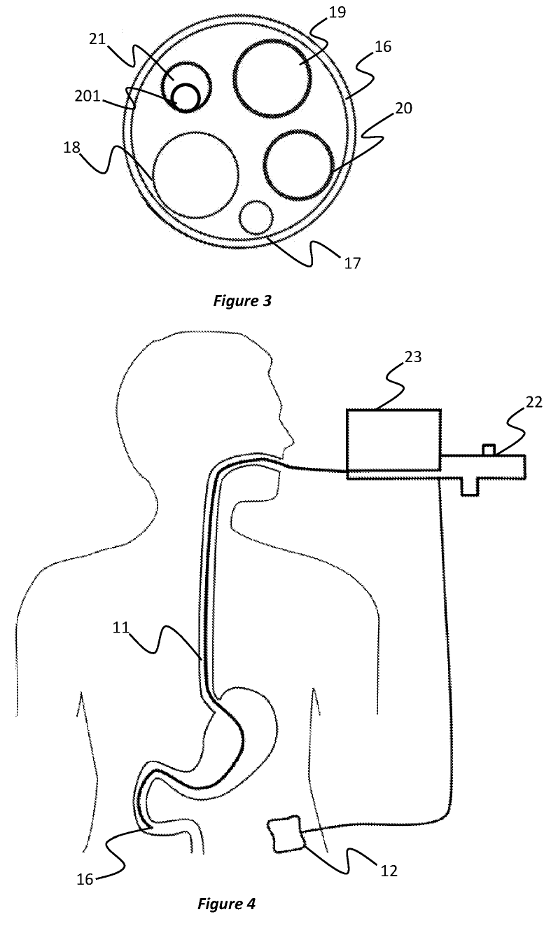

An endoscope controller 22 may be connected to the proximal end of the endoscope 11, as best seen in FIG. 4. The endoscope controller 22 may be configured to be operated by a user to guide the endoscope 11 through the gastrointestinal tract. The controller 22 may comprise a visual display unit 23 for displaying data processed by the processing apparatus 13. Alternatively, the visual display unit 23 may be separate to the controller 22.

An example of a method of using the system 10 will now be described with reference to FIG. 4. The reference electrode 12 is placed on the skin surface of the body. In this example, the reference electrode 12 is placed on the skin surface of the body at the abdomen. It will be appreciated that the reference electrode 12 may be placed on other parts of the body. The endoscope 11 is inserted through the mouth, distal end 16 first, into the gastrointestinal tract of the body with the aid of the endoscope controller 22. In alternative embodiments, the endoscope 11 may be inserted through the rectum. A pulsed electrical current from the stimulator 131 of a known amplitude, duration and amplitude/time relation (wave form) is applied via the first and second electrodes 201, 12 to tissue between the first and second electrodes 201, 12, although in alternative embodiments the electrical current can be applied across this tissue via other electrodes.

The processor 132 is arranged to measure an electrical signal between the first and second electrodes 201, 12 and specifically in this embodiment to measure a voltage between the first and second electrodes 201, 12 in response to the applied electrical current. A bioimpedance value for the tissue between the first and second electrodes 201, 12 can be calculated from the voltage measurement. The bioimpedance value provides a real-time objective measure of inflammation of the wall of the gastrointestinal tract for the region being measured. For example, the present disclosure recognises that a decrease in bioimpedance is synonymous with an increase in permeability and/or leakiness of the wall of the gastrointestinal tract following gut wall inflammation.

Measurements may be made at any point along the gastrointestinal tract and may also be taken at periodic intervals. It will be appreciated that such a method may be employed either alone or in association with other endoscopy procedures. The location of inflamed region in the gastrointestinal tract at the time of measurement may be determined, for example, by the length at which the endoscope has been inserted into the gastrointestinal tract from the mouth or anus and/or the clinician's visual recognition of the anatomy of the gastrointestinal tract at the location that the measurement is made.

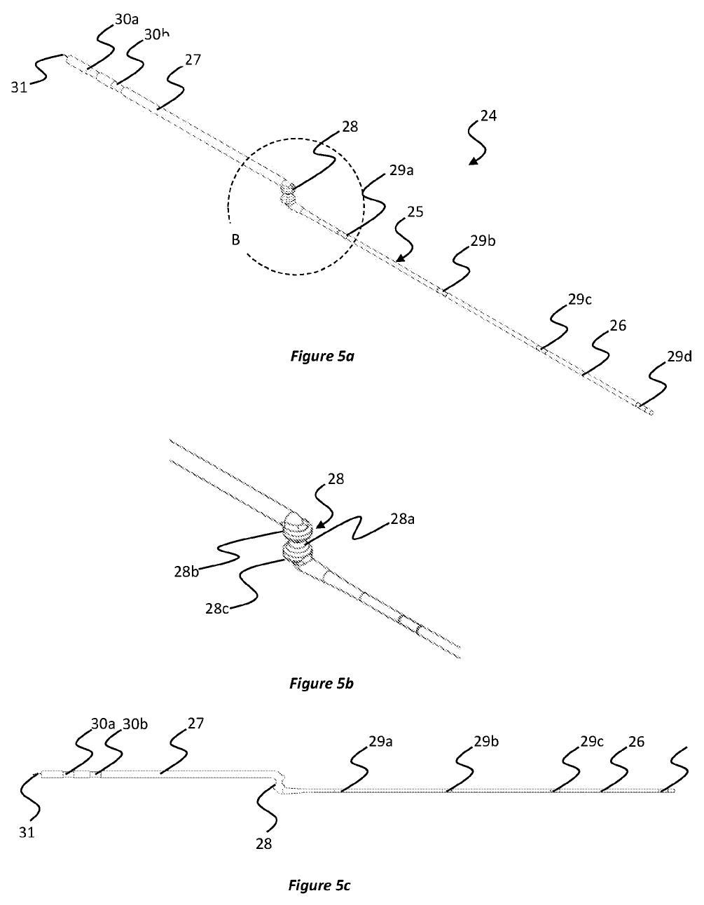

A system for permanent or semipermanent evaluation of IBD according to one embodiment is now described with reference to FIGS. 5a, 5b and 5c. The system is adapted to monitor a particular region of the wall of the gastrointestinal tract that is inflamed or that may be susceptible to inflammation over a period of time. The system comprises a device 24 configured to be implanted into a gastrointestinal tract of a body. The device 24 comprises a flexible lead body 25 that is, for example, formed of silicone or other flexible material. In this embodiment, the device 24 has a generally Z-shaped configuration defined by a distal portion 26, a proximal portion 27 and a transition portion 28 connecting the distal portion 26 and the proximal portion 27.

The body 25 comprises at least one first electrode. In some embodiments, the body 25 comprises a plurality of first electrodes. In the embodiment depicted in FIGS. 5a and 5c, the first electrodes are provided at the distal portion 26 of the body 25 and specifically four electrodes 29a, 29b, 29c, 29d that are spaced longitudinally along the distal portion 26 of the body 25. The body 25 also comprises at least one second (reference) electrode. In some embodiments the body 25 comprises a plurality of the reference electrodes 30a, 30b. In the embodiment depicted in FIGS. 5a and 5c, two reference electrodes 30a, 30b are provided at the proximal portion 27 of the body 25. The electrodes 29a, 29b, 29c, 29d, 30a 30b may be band electrodes formed of any suitable electrically conductive and biocompatible material, such as platinum, platinum iridium alloy or otherwise.

As best seen in FIG. 5b, the transition portion 28 of the body 25 comprises a central portion 28a and first and second swelled portions 28b, 28c on either side of the central portion 28a, connecting to the proximal portion 27 and the distal portion 26 of the body 25, respectively. The central portion 28a has a maximum diameter less than a maximum diameter of the first and second swelled portions 28b, 28c.

A lead wire 31 extends through the lead body 25 and couples the electrodes 29a, 29b, 29c, 29d, 30a, 30b to a processing apparatus. The lead wire 31 may be formed of an electrically conductive material, for example, platinum/iridium. The processing apparatus comprises a stimulator for generating an electrical current and a processor for receiving and processing data from the electrodes 29a, 29b, 29c, 29d, 30a, 30b, for example in a similar manner to the processing apparatus 13 as described above with reference to FIGS. 1 to 4.

A method of implanting the device 24 will now be described, by way of example only. It will be appreciated that other methods may be employed to implant the device 24. Firstly, in this example, a target region of inflammation or suspected inflammation of the wall of the gastrointestinal tract is identified. An O-shaped purse string-suture is placed on the wall of the gastrointestinal tract at a location away from the region of inflammation and a small incision is made in the wall of the gastrointestinal tract within the O-shaped purse string-suture. Other sutures, besides the O-shaped purse string-suture, or other methods of making the incision, may be employed. The device 22 is then inserted through the incision, distal portion 26 first. The distal portion 26 is advanced along the wall of the gastrointestinal tract towards the target region until at least one of the electrodes 29a, 29b, 29c, 29d of the distal portion 26 is adjacent to the target region. When positioned for use, the transition portion 28 of the lead body 25 extends across the incision such that the incision is located around the central portion 28a, and the first and second swelled portions 28b, 28c are located externally and internally of the gastrointestinal tract, respectively. In this position, the distal portion 26 is located wholly within the gastrointestinal tract and the proximal portion 27 is located external to the gastrointestinal tract. In some embodiments, the proximal portion 27 may be located in the peritoneal cavity of the body. The suture is then closed around the central portion 28a of the transition portion 28 sealing the incision and anchoring the device 24 in place. Advantageously, the first and second swelled portions 28b, 28c of the transition portion 28 prevent the device 24 from migrating once the suture is closed. Silicone embedded Dacron patches may be added over the incision and/or along the proximal portion 27 of the lead body 25 to enhance anchoring of the device 24.

An example of a method of using the device 24 will now be described. A pulsed electrical current of a known amplitude, duration and amplitude/time relation (wave form) is applied by the stimulator of the processing apparatus across tissue between the first electrode(s) 29a, 29b, 29c, 29d of the distal portion 26 and the reference electrode(s) 30a, 30b. The electrical current can be applied via combinations of any of the first electrode(s) 29a, 29b, 29c, 29d of the distal portion 24 and any one of the reference electrode(s) 30a, 30b.

The processing apparatus measures one or more electrical signals between the first electrode(s) 29a, 29b, 29c, 29d of the distal portion 26 and/or the reference electrode(s) 30a, 30b, and specifically voltage of one or more electrical signals, in response to electrical current applied by the processing apparatus. A bioimpedance value for the tissue between one or more combinations of the first electrodes 29a, 29b, 29c, 29d of the distal portion 26 and reference electrode(s) 30a, 30b can be calculated from the voltage measurement. The bioimpedance value provides a real-time objective measure of inflammation of the wall of the gastrointestinal tract for the region being measured. Moreover, bioimpedance data of the same region of inflammation or suspected inflammation can be measured over time.

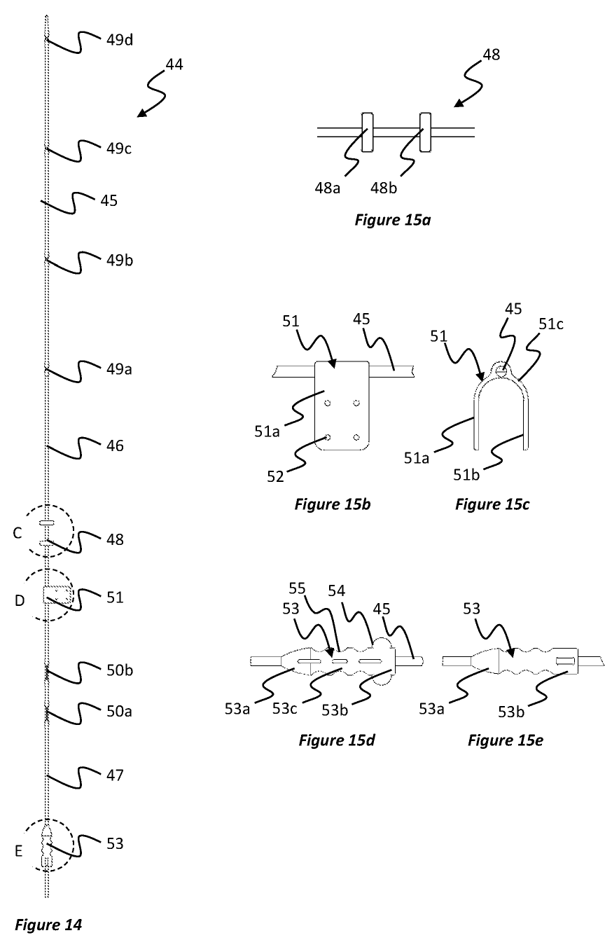

A system for permanent or semipermanent evaluation of IBD according to another embodiment is now described with reference to FIGS. 14 to 15e. The system is similar to the system described above with reference to FIGS. 5a to 5c. However, the system employs a flexible lead body 45 that is generally straight, rather than having a z-shaped configuration, and the system also includes alternative means for securing and anchoring of the lead body.

In more detail, the system comprises a device 44 configured to be implanted into a gastrointestinal tract of a body. The device 44 comprises a flexible lead body 45 that is, for example, formed of silicone or other flexible material. In this embodiment, the device 44 has a generally straight configuration including a distal portion 46, a proximal portion 47 and a transition portion 48 connecting the distal portion 46 and the proximal portion 47. The body 45 includes electrodes 49a, 49b, 49c, 49d, 50a, 50b that are positioned and configured at the distal and proximal portions 46, 47 in a similar manner to the electrodes 29a, 29b, 29c, 29d, 30a, 30b described above with reference to FIGS. 5a to 5c.

As best seen in FIGS. 14 and 15a the transition portion 48 of the body includes two support elements 48a, 48b that are spaced apart along the longitudinal axis of the device 44. In this embodiment, the support elements 48a, 48b are generally planar and have a circular profile (they are generally disk-shaped). When positioned for use, the transition portion 48 extends across the incision in the wall of the gastrointestinal tract such that the planar support elements 48a, 48b are located externally and internally of the gastrointestinal tract, respectively, and rest snugly against opposing surfaces of the wall of the gastrointestinal tract. A purse string suture can be used to close the wall between the two support elements 48a, 48b, for example. In this position, the distal portion 46 is located wholly within the gastrointestinal tract and the proximal portion 47 is located external to the gastrointestinal tract, including in the peritoneal cavity of the body.

To further enhance anchoring of the device 44, as best seen in FIGS. 14, 15b and 15c, a clip 51 (or patch) is provided at the proximal portion 47 of the device 44, spaced from and positioned proximally of the transition region 48. The clip 51 includes two spaced apart flaps 51a, 51b connected by a bridge 51c. The bridge 51c is mounted to the flexible lead body 45. The flaps 51a, 51b are configured to press against opposite external wall surfaces of the gastrointestinal tract at a periphery of the gastrointestinal tract. For example, the flaps may press against wall surfaces adjacent the mesenteric border. The clip 51 is formed from flexible silicone, allowing it to expand and contract with the gastrointestinal tract to accommodate peristaltic movement. The clip 51 includes suture holes 52 to permit suturing of the clip 51 to the wall, although other securing methods such as glue or microneedles may be used.

To yet further enhance anchoring of the device 44, including the flexible lead body 45, as best seen in FIGS. 14, 15d and 15e an abdominal wall anchor 53 is included in the device 44. The anchor 53 is an elongate element that fits around the lead body 45 increasing the diameter of the lead body 45. The anchor 53 is configured to extend across an incision in the abdominal wall. The anchor 53 includes a distal end region 53a that is tapered to assist with insertion into the incision. The anchor 53 also includes a proximal end region 53b having outwardly protruding wings 54 to prevent movement of the entire anchor 53 through the incision. Between the distal and proximal end regions 53a, 53b, a central region 53c is provided around which the incision rests. The central region 53c includes a plurality of channels 55 into which the tissue at the incision can rest for a firmer fit. The anchor 53 can stabilise the device 44 against aggressive bending or movement relative to tissue.

The above described system can be employed as a “closed-loop” system for vagus nerve stimulation (VNS) in the treatment of IBD. There is clinical evidence that VNS is a feasible therapy for the treatment of IBD (see, for example, Bonaz et al., Neurogastroenterol Motil (2016) doi: 10.1111/nmo.12792, 1-6). However, current systems employed for VNS in the treatment of IBD are “open-loop” systems and therefore must be continuously adjusted by the clinician in response to changes in the degree of inflammation associated with IBD. It is envisaged that the implanted device 24 may, for example, be employed with an implanted stimulator and vagus nerve electrode array in a “closed-loop” system to provide real-time monitoring of bioimpedance across the wall of the gastrointestinal tract and therapeutic vagus nerve stimulation, where necessary.

In another example, the present disclosure relates to a method of diagnosing a disorder associated with impaired barrier function in the gastrointestinal tract of a subject. The term “impaired barrier function” is used in the context of the present disclosure to refer to a leaky or permeable gastrointestinal epithelium. For example, impaired barrier function in the gut can refer to a leaky or permeable gastric epithelium. In an example, a leaky or permeable gastrointestinal epithelium results from epithelial cell loss and/or malfunctioning tight junctions.

Various disorders are associated with impaired barrier function. For example, inflammatory disorders can be associated with impaired barrier function. The term “inflammatory disorder” is used in the context of the present disclosure to refer to disorders associated with inflammation of the gut or other regions of the gastrointestinal tract. An example of an inflammatory disorder is inflammatory bowel disease. Other exemplary inflammatory disorders include Crohn's disease, ulcerative colitis, coeliac disease, protein losing enteropathy, non-alcoholic fatty liver disease, non-invasive reflux disease (NERD), gastritis and leaky gut.

Other examples of disorders associated with impaired barrier function include esophagitis, barrett's esophagus, gastrointestinal dysplasia's such as those localized to the esophagus or gut, gastric metaplasia and gastric ulcers. In an example, these disorders may also be characterized as inflammatory disorders.

The term “subject” is used in the context of the present disclosure to refer to any organism suspected of having impaired barrier function and/or an inflammatory disorder. In an example, the subject is a mammal. In one example, the subject is a human. Other exemplary mammalian subjects include companion animals such as dogs or cats, or livestock animals such as horses, cows, poultry and pigs. Terms such as “subject”, “patient” or “individual” are terms that can, in context, be used interchangeably in the present disclosure.

Subjects assessed according to the present disclosure may have symptoms indicative of an inflammatory disorder. For example, a subject may have gastrointestinal symptoms indicative of an inflammatory disorder. Exemplary gastrointestinal symptoms include diarrhea, constipation, nausea, vomiting, flatulence, cramping, bloating, abdominal pain, steatorrhea, rectal bleeding. In another example, a subject assessed according to the present disclosure may present with one or more symptoms selected from the group consisting of fatigue, weakness and lethargy, iron deficiency, anemia, vitamin and mineral deficiency, failure to thrive, delayed puberty, weight loss, bone and joint pain, recurrent mouth ulcers and/or swelling of mouth or tongue, altered mental alertness and irritability, skin rashes such as dermatitis, herpetiformis, easy bruising of the skin and regular reflux.

When performing the methods according to the present disclosure bioimpedance is measured across a subject's gastrointestinal tract wall. For example, bioimpedance can be measured in a gastrointestinal tract wall comprising a columnar epithelium. For example, bioimpedance can be measured in a gut wall of a subject. In another example, bioimpedance is measured in the duodenum of the subject. In another example, bioimpedance is measured in the colon of the subject. In these examples, bioimpedance can measured using an endoscope such as an endoscope according to the present disclosure. Various methods of measuring bioimpedance are discussed above. In an example, measuring bioimpedance can comprise measuring an electrical signal between first and second electrodes, the first and second electrodes being located relative to a body, at least the first electrode being located in a gastrointestinal tract of the body. In this example, the first electrode can be intra-luminal and the second electrode can be extra-luminal.

In another example, bioimpedance is determined by measuring peak voltage. In an example, peak voltage is determined based on about 800 μA of current. In an example, peak voltage is determined based on about 810 μA of current. In an example, peak voltage is determined based on about 820 μA of current. In an example, peak voltage is determined based on about 830 μA of current. In an example, peak voltage is determined based on about 840 μA of current. In an example, peak voltage is determined based on about 850 μA of current. In an example, peak voltage is determined based on about 860 μA of current. In an example, peak voltage is determined based on about 870 μA of current. In an example, peak voltage is determined based on about 880 μA of current. In an example, peak voltage is determined based on about 890 μA of current. In an example, peak voltage is determined based on about 900 μA of current. In another example, peak voltage is determined based on about 910 μA of current. In another example, peak voltage is determined based on about 920 μA of current. In another example, peak voltage is determined based on about 930 μA of current. In another example, peak voltage is determined based on about 940 μA of current. In another example, peak voltage is determined based on about 950 μA of current. In another example, peak voltage is determined based on about 800 μA to 950 μA of current. In another example, peak voltage is determined based on about 820 μA to 900 μA of current. In another example, peak voltage is determined based on about 830 μA to 980 μA of current. In another example, peak voltage is determined based on about 850 μA to 970 μA of current. In another example, bioimpedance is measured using an above referenced method or system.

In another example, bioimpedance is determined by measuring the peak voltage transient produced by a biphasic electrical pulse delivered between intra-luminal and extra-luminal electrodes. In an example, the biphasic pulse has a pulse width of between 10 μs and 500 μs per phase. In an example, the biphasic pulse has a pulse width of between 20 μs and 200 μs per phase. In another example, the biphasic pulse has a pulse width of between 25 μs and 100 μs per phase.

In another example, the biphasic pulse has a pulse width of between 25 μs and 50 μs per phase.

In another example, the biphasic pulse has a pulse width of 25 μs per phase.

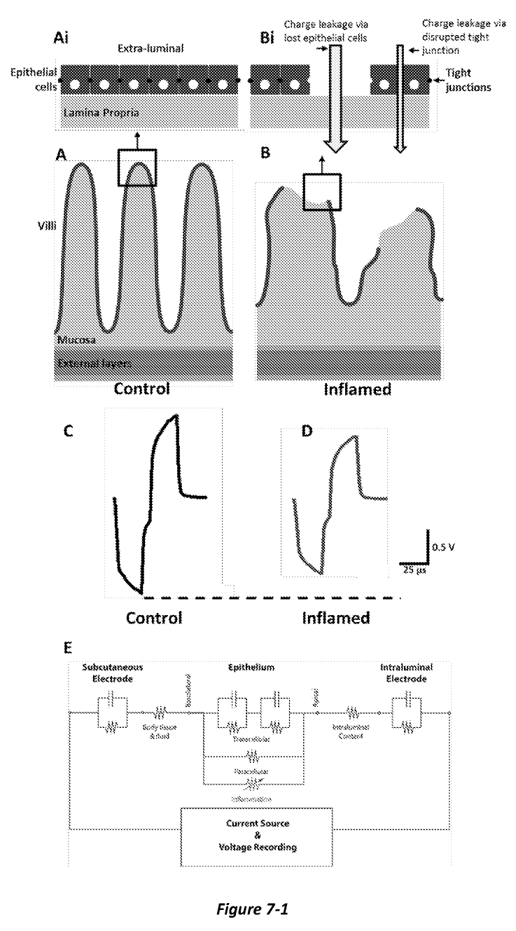

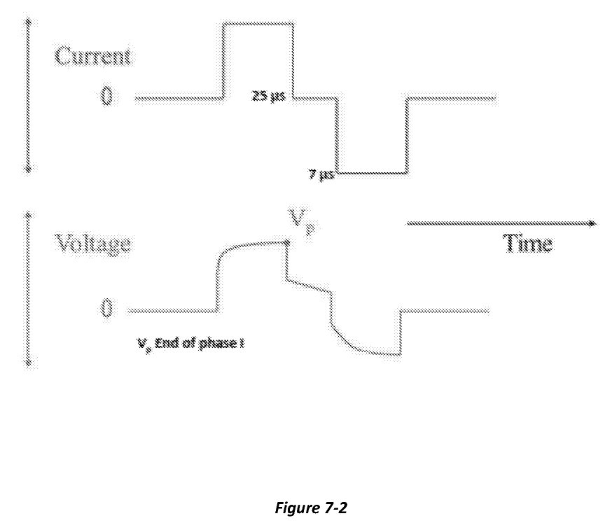

Another exemplary biphasic waveform suitable for use in methods according to the present disclosure is shown in FIG. 7-2. Another example, of measuring bioimpedance is discussed below in the Examples.

In an example, the intra-luminal and extra-luminal electrodes are positioned in the gastrointestinal tract. For example, the intra-luminal and extra-luminal electrodes can be positioned in the gut. In another example, the intra-luminal electrode is positioned in the gastrointestinal tract and the extra-luminal electrode is positioned subcutaneously. In another example, the intra-luminal electrode is positioned in the gut and the extra-luminal electrode is positioned in the duodenum. In another example, the intra-luminal electrode is positioned in the gut and the extra-luminal electrode is positioned subcutaneously.

In an example, at least two, at least three, at least four, at least five, at least six, at least 7, at least 8, at least 9, at least 10 intra-luminal electrodes are used to measure bioimpedance. In another example, at least 20, at least 30, at least 50 intra-luminal electrodes are used to measure bioimpedance. In another example, at least two, at least three, at least four, at least five, at least six, at least 7, at least 8, at least 9, at least 10 extra luminal electrodes are used to measure bioimpedance. In another example, at least 20, at least 30, at least 50 extra luminal electrodes are used to measure bioimpedance. In these examples, electrodes may be positioned at least 0.5 cm apart. In another example, electrodes are positioned at least 1 cm apart. In another example, electrodes are positioned at least 2 cm apart.

In another example, performing the methods of the present disclosure to detect a disorder associated with impaired barrier function in the gastrointestinal tract of a subject, by determining bioimpedance across a subject's gastrointestinal tract wall enables establishment of a diagnostic or prognostic rule based on the bioimpedance. For example, the diagnostic or prognostic rule can be based on the measure of bioimpedance relative to a control. An exemplary control is bioimpedance of a healthy gastrointestinal tract wall. In an example, healthy tissue from the subject being investigated is used to establish control bioimpedance. In another example, the control can be bioimpedance of a healthy subject's gut wall. In these examples, a decrease in bioimpedance relative to a control indicates the presence of a disorder associated with impaired barrier function in the gastrointestinal tract.

In another example, the diagnostic or prognostic rule is based on the application of a statistical and machine learning algorithm. Such an algorithm uses relationships between measures of bioimpedance and disease status observed in training data (with known disease status) to infer relationships which are then used to predict the status of patients with unknown status. An algorithm is employed which provides an index of probability that, for example:
columnar epithelial cell loss and/or tight junction malfunction has occurred in the gastrointestinal tract of a subject;
a subject has an inflammatory disorder;
a subject has inflammatory bowel disease;
a subject has an inflammatory disorder selected from the group consisting of Crohn's disease, ulcerative colitis, coeliac disease, protein losing enteropathy, non-alcoholic fatty liver disease, non-invasive reflux disease (NERD), gastritis and leaky gut.

In another example, the present disclosure relates to a method of allowing a user to determine the status, prognosis and/or treatment response of a subject, the method including (a) receiving data indicating bioimpedance across a subject's gastrointestinal tract wall; b) processing the data to detect epithelial cell loss and/or tight junction malfunction in the subject's gastrointestinal tract; and c) outputting the status, prognosis and/or treatment response of a subject.

In an example, a decrease in bioimpedance relative to a control is used to detect a disorder associated with impaired barrier function in the gastrointestinal tract.

For example, a reduction in bioimpedance of about 2 to 20% or more relative to a control indicates impaired barrier function in the gastrointestinal tract of a subject and/or an inflammatory condition. In another example, a reduction in bioimpedance of about 3 to 15% relative to a control indicates impaired barrier function in the gastrointestinal tract of a subject and/or an inflammatory condition. In another example, a reduction in bioimpedance of about 5 to 10% relative to a control indicates impaired barrier function in the gastrointestinal tract of a subject and/or an inflammatory condition.

For example, a reduction in bioimpedance of at least 5% relative to a control indicates impaired barrier function in the gastrointestinal tract of a subject and/or an inflammatory condition. In another example, a reduction in bioimpedance of at least 6%, 7%, 8%, 9% relative to a control indicates impaired barrier function in the gastrointestinal tract of a subject and/or an inflammatory condition. In another example, a reduction in bioimpedance of at least 10% relative to a control indicates impaired barrier function in the gastrointestinal tract of a subject and/or an inflammatory condition. In another example, a reduction in bioimpedance of at least 15% relative to a control indicates impaired barrier function in the gastrointestinal tract of a subject and/or an inflammatory condition.

In an example, the methods of the present disclosure encompass resolving an inconclusive clinical assessment for inflammatory bowel disease by determining bioimpedance across a subject's gut wall. As used herein, an “inconclusive clinical assessment for inflammatory bowel disease” is inconclusive for inflammatory bowel disease and therefore is not informative for reaching a diagnosis. For example, an inconclusive assessment can refer to a blood test that identifies abnormal sedimentation rate (ESR) and/or C-reactive protein (CRP) levels that are not indicative of inflammatory bowel disease. Other examples include inconclusive stool tests, histology, endoscopic assessments, X-rays, CT scans, MRI scans or a combination thereof. As used herein, the term “resolving” refers to the resolution of an inconclusive clinical assessment for inflammatory bowel disease to determine the clinical status of a subject. For example, the methods of the present disclosure can be performed as an adjunctive test. A test that provides information that adds to or assists in the interpretation of the results of other tests, and provides information useful for resolving an inconclusive earlier assessment may be classified as an adjunctive test. In performing adjunctive testing it is envisaged that bioimpedance can be measured at or about the same time as the other tests. For example, during a single endoscopic session, the gut may be viewed visually, bioimpedance measured and biopsy obtained for histology. However, the various tests may be performed separately.

In another example, the methods of the present disclosure may be performed as a reflexive test. A “reflexive test” refers to a subsequent test (e.g., a second test) that is undertaken based upon the results obtained in a previous test (e.g., a first test). When determining whether a subject has a disorder associated with impaired barrier function in the gastrointestinal tract such as inflammatory bowel disease, assessment of the subject (e.g. blood test, endoscopy, histology) can lead to a desire to perform a further test.

In applying the methods of the present disclosure, it is considered that a diagnostic determination regarding the presence of a disorder associated with impaired barrier function in the gastrointestinal tract can be made based on bioimpedance. However, the diagnostic determination may or may not be conclusive with respect to the definitive diagnosis upon which a treating physician will determine a course of treatment. Put another way, a diagnostic determination obtained using the methods of the disclosure would be understood by one skilled in the art to refer to the process of attempting to determine or identify a disorder associated with impaired barrier function in the gastrointestinal tract. Accordingly, in an example, the methods of the present disclosure can be used to provide assistance in making an assessment of a pre-clinical determination regarding the presence an inflammatory disorder. This would be considered to refer to making a finding that a subject has a significantly enhanced probability of having the inflammatory disorder.

### EXAMPLES

### Example 1—Electrode Array, Gut Wall Bioimpedance and Inflammation

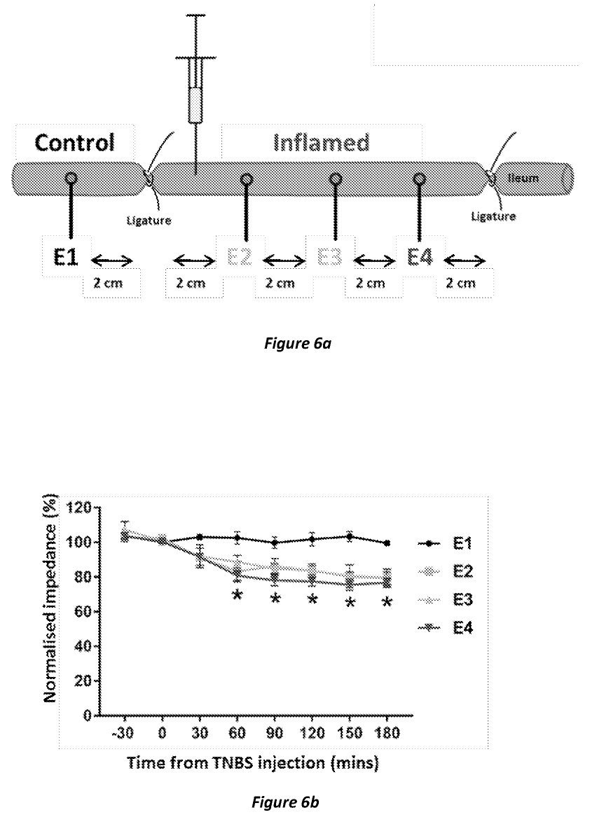

With reference to FIG. 6a, four platinum electrodes were stitched into a segment of a small intestine. The inflammatory agent, trinitrobenzenesulphonic acid (TNBS), was injected into the relevant region of the gut for a duration of 3 hours to induce inflammation. Electrode 1 (E1) was placed in a region of gut that did not receive TNBS (control), while the region containing electrodes 2-4 was isolated using ligatures (indicated by ‘rope’) and injected with TNBS. The voltage transient between intraluminal electrodes and a subcutaneous electrode return was measured using 25 μs phase with 8 μs interphase gap biphasic pulses (monopolar) delivered at 931 μA.

Following implantation into the ileum, Zₜₒₜₐₗ for all electrodes was significantly elevated (1241±170 mV n=24 electrodes; n=6 rats) compared to saline (495±36.5 mV). The increase in Vₜₒₜₐₗ was presumed to be dominated by the transmural impedance including the presence of epithelial cells and associated tight junctions (FIG. 7-1A-E). Therefore, any increases in transmural permeability would be expected to result in a drop in Vₜₒₜₐₗ towards pre-implantation saline values. Consequently, normalised Vₜₒₜₐₗ is hereafter referred to as normalised gut wall impedance.

Throughout the testing period, normalised gut wall impedance in the non-inflamed region of ileum (E1), remained stable (P>0.05; n=6 rats/electrodes, FIG. 6b). Following TNBS injection, an electrical current was applied to the electrodes and impedance data was measured based on the applied electrical current. An analysis of the impedance data indicated a significant decrease in impedance for the second to fourth electrodes E2 to E4 after 120 minutes, relative to impedance data of the first electrode E1. In particular, normalised gut wall impedances in inflamed regions (E2-4) rapidly reduced and were significantly less than the control E1 at 60 mins and remained so for the duration of the experiment (180 min; FIG. 6b; P<0.05; n=6). There were no significant differences between inflamed regions (E2, E3 and E4) following TNBS injection (P≥0.05; FIG. 6b).

### Histological Scoring of Inflammatory Damage

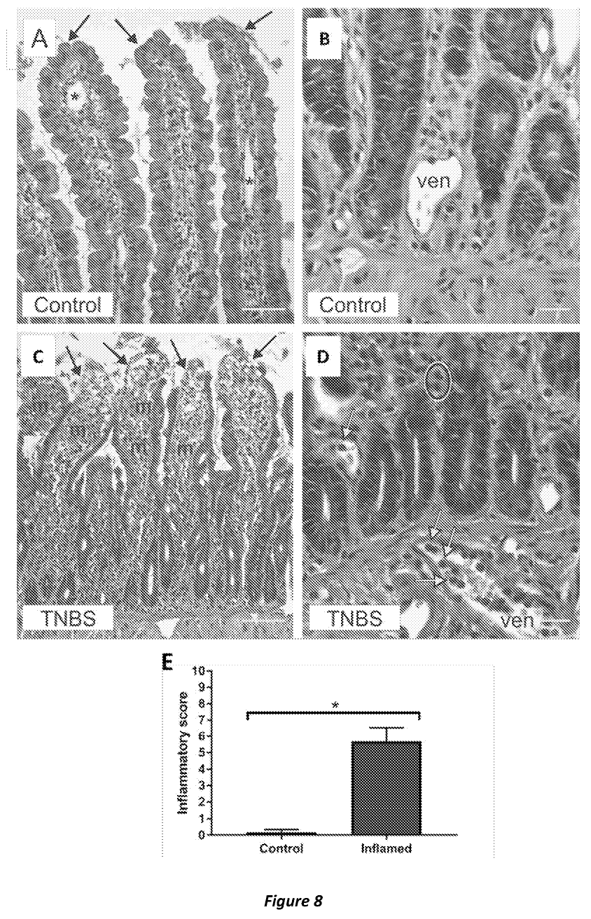

Histological scoring of control (adjacent to electrode position E1) and inflamed tissue (adjacent to electrode positions E2-E4) was based on parameters described in Table 1. FIG. 8 A, C: Images show comparison of the villi. In A, the surface epithelium was intact at the tips of the villi (arrows). Lymphatic vessels (asterisks) were apparent but there were no red blood cells in the connective tissue spaces. In C, the villi were shortened and the base of the mucosa can be seen in the same field as villus tips. The surface epithelium was lost from the tips of the villi (arrows). Numerous regions of microhemorrhage were seen in the villi (m). The lack of inflammation indicates that the implantation of the Pt ball electrodes did not invoke an inflammatory response.

There was marked accumulation of neutrophils in the venules (ven; indicated by arrows in FIG. 8 D), which was not seen in control tissue (C). The crypts were intact in both control and TNBS treated tissue. Eosinophils (circled) were more numerous in inflamed tissue (D), than in control (B). FIG. 8 E: Histologically scored tissue damage was significantly more prevalent within inflamed tissue (n=18), than in control (E1; n=6). Data shows mean (±S.E.M), and differences were considered significant for P<0.05. Scale bars in A-B represent 50 μm; Ai-Bi: 20 μm.

Decreased Normalised Gut Wall Impedance Correlates with Immune Cell Infiltration

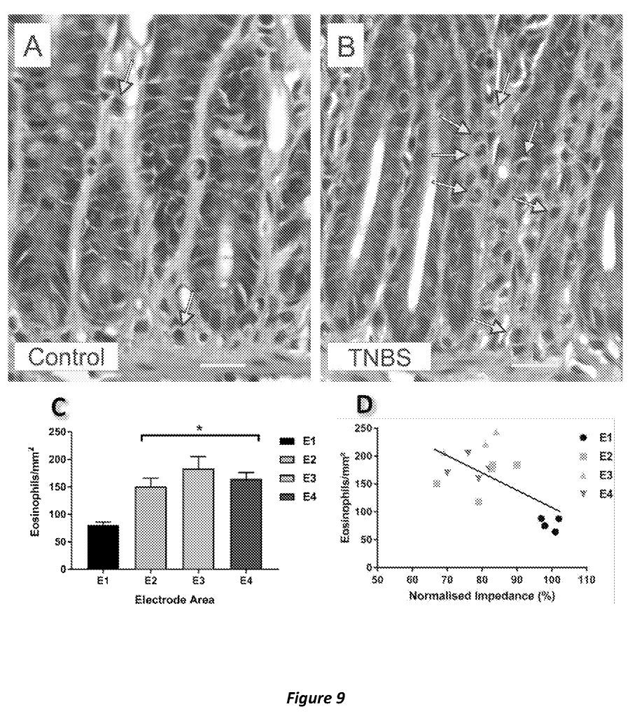

Small numbers of eosinophils were quantified in control mucosa (E1; n=6 segments of control tissue) (FIG. 9A), in the submucosa (75.0±18.2 cells/mm²) and in the circular muscle (2.4±1.6 cells/mm²), but none were observed in the longitudinal muscle. At 3 h following TNBS injection (FIG. 9B), the density of eosinophils was significantly greater within the mucosa of the inflamed segments of ileum (E2-E4; n=6 rats; n=18 pieces of ileum; P=0.001; FIG. 9C). There was no evidence of eosinophil infiltration within the submucosa and the longitudinal and circular muscle (P≥0.05; n=18). There was a significant correlation between eosinophil infiltration in the mucosa and normalised gut wall impedance at 3 h post TNBS injection (FIG. 9D; P=0.006; R=−0.66).

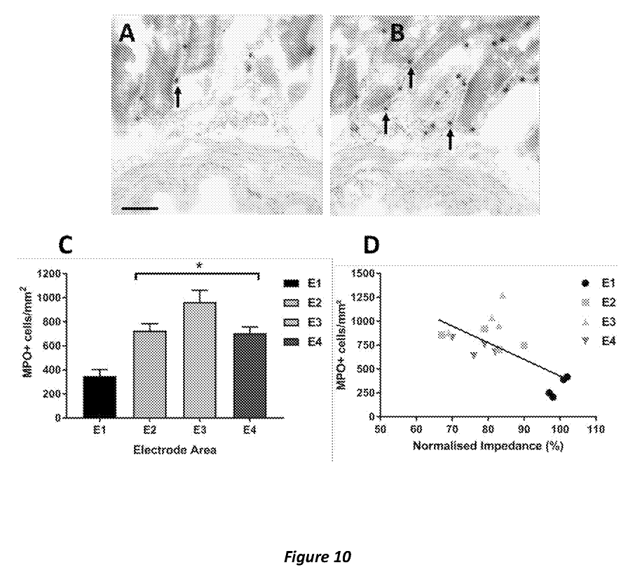

Neutrophils were identified by their myeloperoxidase activity (MPO+; FIG. 10A, B, indicated by arrows). MPO+ cells were observed in control tissue (n=6 segments of control tissue), including in smooth muscle (5.6±1.6 cells/mm²), submucosa (216.6±42.6 cells/mm²) and mucosal tissue (350.4±52.9; FIG. 10A). Three hours following TNBS injection (n=6 rats; n=18 segments of inflamed ileum), there was no infiltration of MPO+ cells into smooth muscle (P≥0.05) or submucosa (P≥0.05). However, there was significant infiltration of neutrophils into the mucosa (P<0.0001; FIG. 10B), with no significant difference between neutrophil densities in different electrode locations (E2, E3 and E4; FIG. 10C; P≥0.05). The density of neutrophils in the mucosa was significantly correlated (P=0.005; R=−0.65; n=16) with normalised gut wall impedance measurements at 3 h post TNBS injection (FIG. 10D).

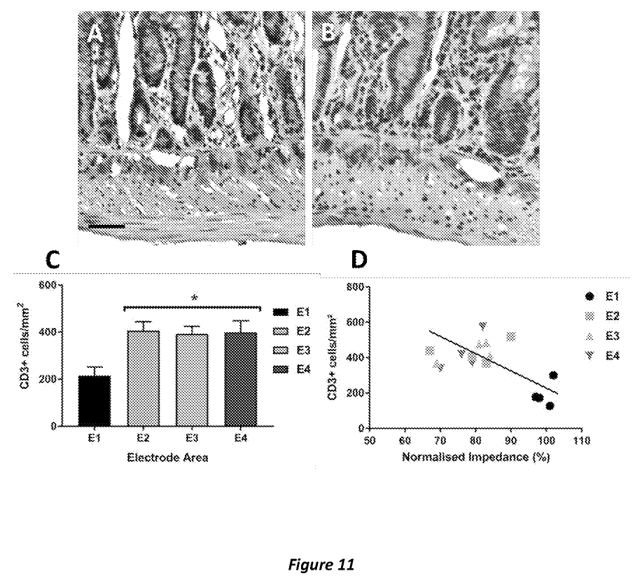

Very few CD3+ T-cells were seen in control tissue (FIG. 11A; E1; n=6 segments of control tissue; smooth muscle: 1.6±1.2 cells/mm²; submucosa layer: 40.6±13.6 cells/mm²). CD3+ cells were observed in the mucosal layer of control tissue (FIG. 11A, indicated with arrow head). Following TNBS injection (FIG. 11B; n=18 sections of inflamed ileum), there was no infiltration of CD3+ cells into the smooth muscle layer or the submucosa (P≥0.05), while a significant infiltration of CD3+ cells was evident in the mucosa (P=0.008; FIG. 11C). The densities of CD3+ cells were similar at electrode locations E2, E3 and E4 (FIG. 11C; P≥0.05). The density of CD3+ cells in the mucosa was significantly correlated (P=0.023; R=−0.57) with normalised gut wall impedance measurements at 3 h post TNBS injection (FIG. 11D).

### Example 2—Dose-Dependent Decrease in Transmural Impedance Detected in Awake Rats Following Inflammation

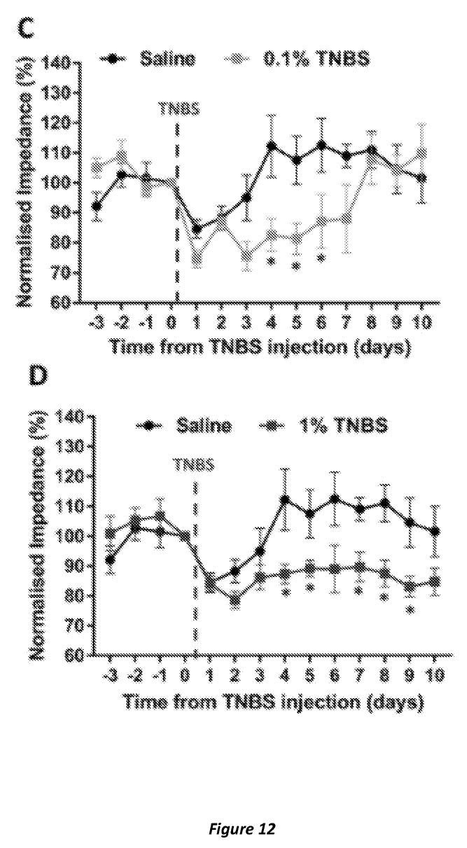

A four-ring platinum electrode array was implanted in the ileus of rats (n=4 rats/group; n=12 electrodes per time point) with saline, 0.1% or 1% TNBS being administered two weeks following implantation. Impedance measurements were then taken daily for 10 days. Following 0.1% TNBS injection, impedance was significantly less than saline control between days 4-6, after which it returned to baseline levels (FIG. 12C). Following 1% TNBS injection, impedance was significantly less than saline control between days 4-9, after which it returned to baseline levels (FIG. 12D).

### Example 3—Decrease in Transmural Impedance in Sheep Ileum was Similar to that Seen in Rat

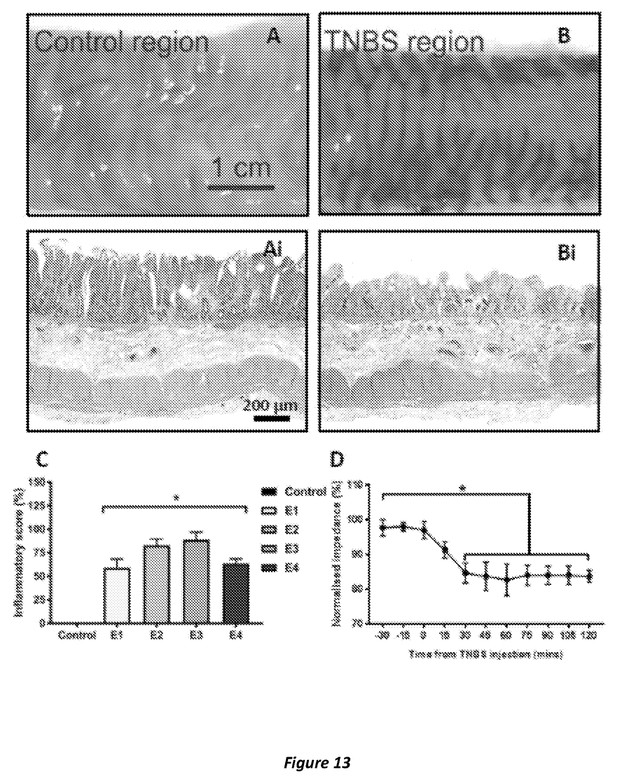

A single dose of 0.1% trinitrobenzene-sulphonic acid injection (0.1% TNBS in 50% ethanol) was injected into the ligated lumen of Marino sheep (20 cm segment, 30 ml) into which a PT electrode array was inserted. Transmural impedance was measured using 25 μs phase with 8 μs interphase gap biphasic pulses (monopolar) delivered at 860 μA. Transmucosal impedance (Ohms) was calculated from the mean peak voltage at the end of the first phase of the stimulus divided by the current amplitude (0.86 mA). Macroscopic view shows vasodilation (FIG. 13) and significant inflammatory-associated damage to villi (FIG. 13Bi, C; P<0.05; n=3 sheep) compared to control (FIG. 13A, Ai, C). Significant, ˜20% decrease in transmucosal impedance was seen across all electrodes in inflamed region (n=3 sheep; 12 electrodes P<0.05; FIG. 13D).

### Example 4—Vagus Nerve Stimulation Alleviates Ileitis

A therapeutic stimulating array was implanted on the vagus nerve (VN) of rat. At 2 weeks following implantation surgery, the ileum of rats was injected with TNBS. Awake, freely moving rats (n=3/group) received active stimulation (10 Hz; 1.6 mA; 3 hours/day for 5 days). Untreated rats (n=3) received no stimulation. Histology at 4 hours post TNBS injection was examined to determine the extent of tissue damage prior to stimulation. At 5 days post TNBS injection, animals were euthanased and processed for histology. Tissue was scored for signs of inflammatory-induced damage. Data is expressed as mean+standard error of mean (SEM) and differences considered significant for P<0.05.

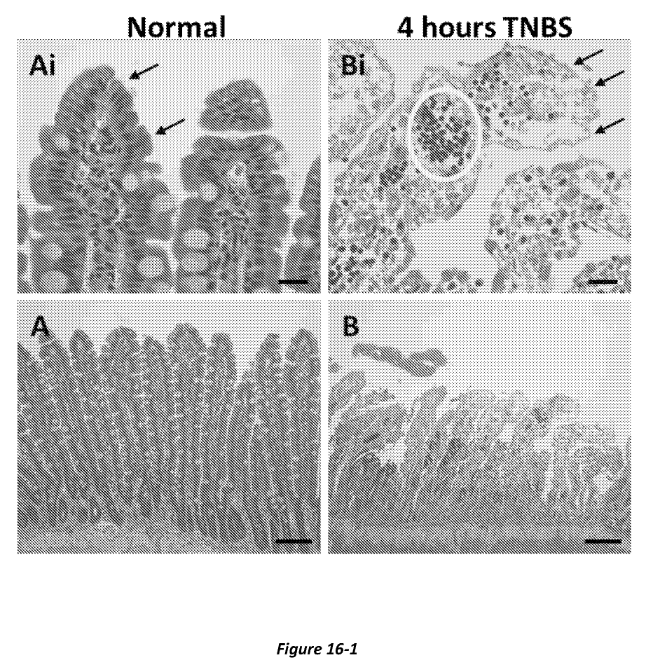

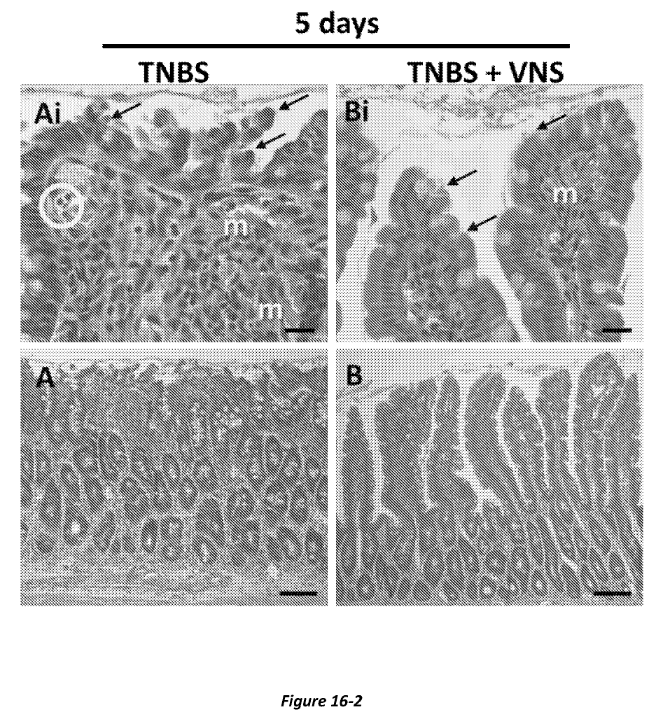

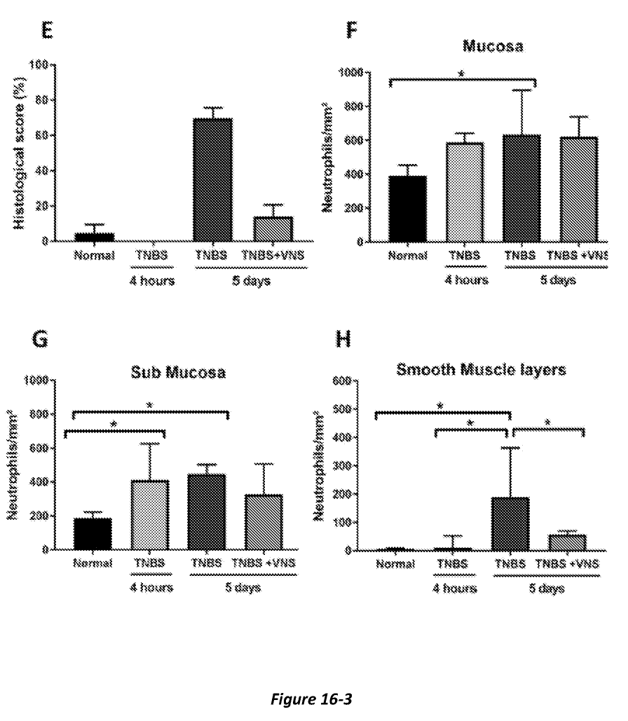

FIG. 16 shows that normal ileum (A, Ai) had undamaged, intact surface epithelium (arrows, Ai). Scale bars in A-B: 100 μm and in Ai-Bi: 20 μm. FIGS. 16-1B, Bi and 16-2A, Ai provides an overview of histology 4 hours and 5 days following TNBS injection in unstimulated tissue. 4 hours following TNBS injection extensive epithelial cell loss (arrow, 16-1Bi) and leukocyte infiltration (circle, Bi) was observed. 5 days following TNBS injection, unstimulated tissue had extensive damage to villi (16-2 Ai, arrows), leukocyte infiltration in venules (circle, Ai), microhemorrhages (m, Ai) and villi architecture were severely disrupted. FIG. 16-1 B, Bi provides an overview of histology 4 hours (the time that active VN stimulation was applied) and FIG. 16-2 B, Bi shows 5 days following TNBS injection in stimulated tissue. In VN stimulated tissue, signs of histological damage was reduced. Villi were long and undamaged. Surface epithelium were intact (arrows, Di) and, although reduced in number compared with unstimulated animals, some microhemorrhages were observed (16-2 B, Bi; m). Histologically scored damage was also more prevalent within inflamed tissue (FIG. 16-3E).

Neutrophils (indicated by myeloperoxidase activity) were also quantified within ileum tissue (FIG. 16-3 F-H). TNBS induced a significant increase in neutrophils within mucosa (F), submucosa (G) and muscle layers (H). However, following VN stimulation, neutrophils were significantly reduced in muscle layers (H) and were no different from normal within mucosa (F) and submucosa (G) layers. Differences between groups (P<0.05) were tested using non-parametric Kruskal-Wallis and Dunn's post hoc test.

### Example 5—Materials and Methods

### Rats and Surgical Procedures

All experiments were approved by the Animal Research and Ethics Committee of the Bionics Institute, complied with the Australian Code for the Care and Use of Animals for Scientific Purposes (National Health and Medical Research Council of Australia) as well as the United States Army Medical Research and Material Command Animal Care and Use Review Office, protocol SSC-7486.02. Rats were allowed ad libitum access to standard chow, water and fresh food and were kept on a 12 hour light/dark cycle. Prior to surgery, male Sprague Dawley rats were fasted overnight to reduce the amount of content in the gut. On the day of surgery, animals were anaesthetized (2% isoflurane in 1.0% oxygen; pre-operative analgesia Carprofen 50 mg/kg sub cutaneous) before having their abdominal cavity opened and an 8 cm segment of ileum selected approximately 30 cm proximal to the ileum-caecum junction. Four platinum (Pt) ball electrodes (1.2 mm diameter; surface area 3.2 mm²) placed 2 cm apart were inserted into the lumen and secured with sutures (7′0 silk, Ethicon; FIG. 6a). An 18 gauge needle placed subcutaneously acted as a return electrode. A 6 cm segment of ileum containing three Pt electrodes (E2-E4) was isolated using ligatures, while electrode 1 (E1) was placed 2 cm proximal to the ligated area. Following ligation, 30 minutes of baseline voltage transient measurements (see next section) were generated from all electrodes (E1-E4). Inflammation was induced within this ligated area by injecting TNBS (1 ml of 0.1% dilution in 50% ethanol, Sigma) into the lumen.

### Gut Wall Impedance Monitoring

Gut wall impedance was monitored using biphasic current pulses passed between the intra- and extra-luminal electrodes. The peak voltage at the end of the first phase of the current pulse (total voltage Vₜₒₜₐₗ, FIG. 7-1C, D) was measured. A 25 μs per phase biphasic current pulse (7 μs interphase gap) was used to minimize the charging of the electrode-electrolyte interface. This measurement was used to generated impedances for all data sets described (FIGS. 6b, 7-1CD, 9D, 10D, 11D, 12 CD, 13D) The current used in this study was 931 μA which was chosen to produce a peak voltage transient of ˜500 mV (range: 398-543 mV) when the Pt balls electrodes were placed in sterile 0.9% saline. Following each acute experiment, the Pt balls were explanted, cleaned and retested in saline. There was no significant difference between pre- and post-experiment in vitro measures (paired T-test; P≥0.05).

Triplicate measurements were taken from each intra-luminal electrode at T=−30 mins and T=0 prior to TNBS injection to establish a baseline. Following TNBS injection, triplicate measurements were taken at T=30, T=60, T=90, T=120, T=150 and T=180.

### Histology and Staining

Rats were euthanized (300 mg/kg Lethabarb, intracardial injection) at 3 h post TNBS injection and segments of the ileum adjacent to electrodes E1-E4 were removed, placed into cold PBS and cut longitudinally along the mesenteric border, pinned out onto balsa boards and divided in half. One half was placed in fixative (2% formaldehyde plus 0.2% picric acid in 0.1 M sodium phosphate buffer) overnight, embedded in paraffin, sectioned (5 μm), stained with hematoxylin and eosin (H&E) and mounted with DPX. The other half of the tissue was prepared for myeloperoxidase (MPO) staining that reveals neutrophils. Tissue was placed in ice cold 100% ethanol (10 min), washed in cold 0.1 M sodium phosphate buffer (PBS; 3×5 min), then cryoprotected overnight (30% sucrose in PBS). Tissue was immersed overnight in 50% optimal cutting temperature (OCT) medium and 50% sucrose, frozen (20° C.) and sectioned (14 μm). Sections were placed in freshly made Hanker-Yates solution (10 min in 0.003% H₂O₂ with 0.06 mg/mL Hanker-Yates reagent, Polysciences, Warrington, Pa., USA, in PBS), washed then immersed in methyl green aqueous solution (2% Sigma), dehydrated (100% ethanol) and mounted with DPX.

### Immunohistochemistry

Paraffin sections were dewaxed with xylene, graded ethanol and tap water. The sections underwent antigen retrieval (10 mM sodium citrate, 0.05% Tween20, pH 6 and 1% hydrogen peroxide in PBS heated to 60° C. for 10 mins). Sections were incubated with anti-CD3 (1:200 in 10% normal horse serum, NHS, Dako Cytomation), a cytotoxic T-cell marker, overnight at 4° C. Sections were thoroughly washed (0.1M PBS) and the secondary antibody applied (Biotinylated goat anti rabbit 1:500 in 10% NHS, Dako E0432) for 1 h at room temperature. Following washing, (0.1M PBS), streptavidin coupled to horse radish peroxidase (1:1000 in 10% NHS, Dako P0397) was applied, washed (0.1M PBS) and immersed in 3,3′-diaminobenzidine (DAB) (made as per instructions, Dako cat no. #K3468) for 3 min. Slides were counter-stained with Harris hematoxylin (5 sec), washed (distilled water) dehydrated in 100% ethanol and xylene, and mounted in DPX.

### Histological Scoring of Inflammation

Histopathologist (J.B.F), blinded to experimental conditions, used H&E stained sections to evaluate the degree of inflammation at each electrode site (E1-E4). Histological changes were on a scale of 0-3 for the assessment of the extent of damage to the mucosa and the degree (or depth) of damage to the mucosa, and on a scale of 0-2 for assessment of the numbers of leukocytes within venules and the extent of hemorrhage within villi. Scores were out of a total of 10 (Table 1).

### Cell Counting

Quantification of key leukocyte populations is a widely accepted clinical indicator of inflammation in the ileum (Pontell et al. (2009) Virchows Arch. 455:55-65). Eosinophilic granulocytes were identified morphologically using H & E stained sections, by their distinctive nucleus (identified by arrows in FIG. 9A-B). Myeloperoxidase activity was used to identify neutrophils (identified in FIG. 10A-B by arrows). Antibodies to CD3, which identifies a cluster of T-cell receptors on cytotoxic T-lymphocytes were also counted within H&E sections (identified in FIG. 11A-B by arrows). With the observer blinded to the tissue identity, positive cells were counted at objective ×40, across 10 fields of view at each electrode position (E1-E4) using a Zeiss Axioplan II microscope within the following layers: longitudinal smooth muscle, circular smooth muscle, sub-mucosa and mucosa. Images of the total field of view were generated (Axiovision software, Zeiss, Germany), the area of the total field of view for each layer of tissue was measured using ImageJ, and the total density of cells per mm² was calculated and analysed.

### Statistical Analysis

Voltage transient data was converted into an impedance value using Ohms law. Gut wall impedance values were normalised to the baseline measurements (average of data taken at T=−30 and T=0 minutes prior to TNBS injection). A two-way repeated measures ANOVA (Electrode×Time) was used to test for differences and interactions, and Tukey's post hoc test used where appropriate. Infiltration of leukocytes was assessed using a one-way ANOVA and a Tukey's post hoc test. Normalised gut wall impedances were correlated with leukocyte infiltration and R squared and two tailed P values generated. A P-value of P<0.05 was accepted as statistically significant, and data is expressed as mean+standard error of mean (SEM). GraphPad Prism 4 was used for all analysis (GraphPad Software, USA).

The present application claims priority from AU 2016903957 filed 29 Sep. 2016, the disclosures of which are incorporated herein by reference.

It will be appreciated by persons skilled in the art that numerous variations and/or modifications may be made to the above-described embodiments, without departing from the broad general scope of the present disclosure. As one example, according to embodiments of the present disclosure, rather than calculating bioimpedance by measuring changes in voltage or voltage drops between electrodes for a constant current stimulation signal, a stimulation signal of a known voltage may be applied by the stimulator of the processing apparatus. In these embodiments, the processor may measure electrical current passing between the first and second electrodes in response to the applied voltage. The present embodiments are, therefore, to be considered in all respects as illustrative and not restrictive.

### REFERENCES

Bonaz et al., Neurogastroenterol Motil (2016) doi: 10.1111/nmo.12792, 1-6
Pontell et al. (2009) Virchows Arch. 455:55-65

1. A method of identifying or assessing inflammatory bowel disease, the method comprising:
measuring an electrical signal between first and second electrodes, the first and second electrodes being located relative to a body, at least the first electrode being located in a gastrointestinal tract of the body; and
determining one or more characteristics of inflammatory bowel disease based on the measured electrical signal.

2. The method of claim 1, wherein the one or more characteristics comprises leakiness of the gastrointestinal tract.

3. The method of claim 1 or 2, wherein the one or more characteristics comprises permeability of the gastrointestinal tract.

4. The method of claim 1, 2 or 3, wherein the one or more characteristics comprises inflammation of a wall of the gastrointestinal tract.

5. A method of identifying or assessing gut inflammation comprising:
measuring an electrical signal between first and second electrodes, the first and second electrodes being located relative to a body, at least the first electrode being located in a gastrointestinal tract of the body; and
determining an inflammation of a wall of the gastrointestinal tract based on the measured electrical signal.

6. The method of any one of the preceding claims, wherein the second electrode is located externally of the gastrointestinal tract.

7. The method of any one of the preceding claims, wherein the measured electrical signal is indicative of bioimpedance of tissue between the first and second electrodes.

8. The method of any one of the preceding claims, further comprising determining bioimpedance of tissue between the first and second electrodes based on the measured electrical signal.

9. The method of claim 8, further comprising determining the one or more characteristics based on the determined bioimpedance.

10. The method of any one of the preceding claims, wherein the method further comprises applying an electrical current across tissue between the first and second electrodes, and wherein the measuring of the electrical signal is in response to the applied electrical current.

11. The method of claim 10, wherein the electrical current is applied via the first and second electrodes.

12. The method of claim 10 or 11, wherein the electrical current is applied via one or more additional electrodes.

13. A system for identifying or assessing inflammatory bowel disease or gut inflammation, the system comprising:
a device insertable or implantable into a gastrointestinal tract of a body, the device having at least one first electrode;
a second electrode; and
a processing apparatus configured to determine one or more characteristics of inflammatory bowel disease or gut inflammation based on an electrical signal measured between the at least one first electrode and the second electrode.

14. The system of claim 13, wherein the one or more characteristics comprises inflammation of a wall of the gastrointestinal tract.

15. The system of claim 13 or 14, wherein the second electrode is configured to locate externally of the gastrointestinal tract.

16. The system of claim 13 or 14, wherein the second electrode is configured to locate in the peritoneal cavity of the body.

17. The system of claim 13 or 14, wherein the second electrode is configured to locate at a skin surface of the body.

18. The system of any one of claims 13 to 17, wherein the measured electrical signal is indicative of bioimpedance of tissue between the at least one first electrode and the second electrode.

19. The system of any one of claims 13 to 18, wherein the processing apparatus is configured to determine bioimpedance of tissue between the at least one first and the second electrode based on the measured electrical signal.

20. The system of claim 19, wherein the processing apparatus is configured to determine the one or more characteristics based on the determined bioimpedance.

21. The system of any one of claims 13 to 20, wherein the processing apparatus is further configured to apply an electrical current across tissue between the at least one first electrode and the second electrode, and wherein the measured electrical signal is in response to the applied electrical current.

22. The system of claim 21, wherein the electrical current is applied via the at least one first electrode and the second electrode.

23. The system of claim 21 or 22, wherein the electrical current is applied via one or more additional electrodes.

24. The system of any one of claims 21 to 23, wherein the processing apparatus comprises a stimulator, said stimulator being configured to apply the electrical current.

25. The system of any one of claims 13 to 24, wherein the at least one first electrode comprises a plurality of first electrodes and the processing apparatus is configured to determine one or more characteristics of inflammatory bowel disease or gut inflammation based on a plurality of electrical signals measured between the second electrode and different first electrodes.

26. The system of claim 13, wherein the insertable device comprises an endoscope, the endoscope having a flexible body with a proximal end and a distal end.

27. The system of claim 26, wherein the endoscope comprises any one or more of a channel for the supply of gas and/or liquid into the gastrointestinal tract, an instrument channel, an optical channel, a light channel and an electrode channel.

28. The system of claim 27, wherein the optical channel comprises a camera module for transmitting images from the distal end of the endoscope to the processing apparatus.

29. The system of claim 27 or 28, wherein the light channel comprises a light source for transmitting light to the distal end of the endoscope.

30. The system of any one of claims 27 to 29, wherein the at least one first electrode is provided in the electrode channel at or adjacent the distal end of the endoscope.

31. The system of claim 13, wherein the implantable device comprises a flexible lead body having a distal portion, a proximal portion and a transition portion connecting the distal portion and the proximal portion, wherein the distal portion is configured to be implanted in the gastrointestinal tract, and wherein the proximal portion is configured to be located external of the gastrointestinal tract.

32. The system of claim 31, wherein the transition portion comprises a central portion and first and second swelled portions on either side of the central portion, the first and second swelled portions connecting the proximal and distal portions, respectively.

33. The system of claim 32, wherein the central portion has a maximum diameter less than a maximum diameter of the first and second swelled portions, wherein the first and second swelled portions are configured to prevent migration of the device from an implantation site when positioned for use.

34. The system of claim 33, wherein the lead body is formed of silicone or other flexible material.

35. The system of any one of claims 31 to 34, wherein lead body is of a generally Z-shaped configuration.

36. The system of claim 31, wherein the transition portion comprises two support elements spaced apart along the lead body to contact an internal surface and an external surface, respectively, of a wall of the gastrointestinal tract.

37. The system of any one of claims 31 to 36, wherein the proximal portion comprises a clip configured to mount over external wall surfaces of the gastrointestinal tract.

38. The system of any one of claims 31 to 37 wherein the proximal portion comprises an anchor configured to locate in the abdominal wall.

39. The system of any one of claims 31 to 38, wherein the at least one first electrode is provided at the distal portion of the lead body.

40. The system of any one of claims 31 to 39, wherein the second electrode is provided at the proximal portion of the lead body.

41. The system of any one of claims 31 to 40, further comprising a lead wire extending through the lead body for coupling the first and second electrodes to the processing apparatus.

42. The system of claim 41, wherein the lead wire is formed of any suitable electrically conductive and biocompatible material.

43. The system of claim 42, wherein the lead wire is formed of platinum, platinum iridium alloy or otherwise.

44. The system of any one of claims 13 to 43, wherein the first and second electrodes are band electrodes.

45. The system of claim 44, wherein the first and second electrodes are formed of electrically conductive and biocompatible material.

46. The system of claim 45, wherein the first and second electrodes are formed of platinum, platinum iridium alloy or otherwise.

47. The system of any one of claims 13 to 46 wherein the device is temporarily or chronically insertable or implantable into the gastrointestinal tract of the body.

48. A method comprising:
inserting or implanting a device into a gastrointestinal tract of a body, the device comprising at least one first electrode;
locating a second electrode relative to the body;
coupling a processing apparatus to the at least one first electrode and the second electrode, the processing apparatus being configured to determine an inflammation of a wall of the gastrointestinal tract based on an electrical signal measured between the at least one electrode and the second electrode.

49. The method of claim 48, wherein the second electrode is located externally of the gastrointestinal tract.

50. The method of claim 48 or 49, wherein the second electrode is located in the peritoneal cavity of the body.

51. The method of claim 48 or 49, wherein the second electrode is located on a skin surface of the body.

52. The method of any one of claims 48 to 51, wherein inserting or implanting the device comprises anchoring the device to the wall of the gastrointestinal tract at or adjacent a region of gut wall inflammation.

53. Method of detecting a disorder associated with impaired barrier function in the gastrointestinal tract of a subject, the method comprising determining bioimpedance across a subject's gastrointestinal tract wall and detecting impaired barrier function based on bioimpedance.

54. The method of claim 53, wherein impaired barrier function is detected based on a decrease in bioimpedance relative to a control.

55. Method of detecting an inflammatory disorder in a human subject, the method comprising determining bioimpedance across a subject's gastrointestinal tract wall and detecting a decrease in bioimpedance relative to a control.

56. The method of any one of claims 53 to 55, wherein the gastrointestinal tract wall is the gut wall.

57. Method of resolving an inconclusive clinical assessment for inflammatory bowel disease, the method comprising determining bioimpedance across a subject's gut wall, wherein a decrease in bioimpedance relative to a control indicates the presence of inflammatory bowel disease.

58. The method of claim 53 or 54, when used for detecting an inflammatory disorder.

59. The method of claim 58, wherein the inflammatory disorder is inflammatory bowel disease.

60. The method of claim 58, wherein the inflammatory disorder is selected from the group consisting of Crohn's disease, ulcerative colitis, coeliac disease, protein losing enteropathy, non-alcoholic fatty liver disease and non-invasive reflux disease (NERD), gastritis or leaky gut.

61. The method of any one of claims 56 to 60, wherein intra and extra-luminal electrodes are positioned in the subject's gut wall and bioimpedance is determined by measuring an electrical signal between intra-luminal and extra-luminal electrodes.

62. The method of claim 61, wherein the measured signal is based on a biphasic electrical pulse delivered between the intra-luminal and extra-luminal electrodes.

63. The method of claim 61 or claim 62, wherein the electrical signal is current.

64. The method of claim 61 or claim 62, wherein the electrical signal is voltage.

65. The method of claim 64, wherein the measured voltage is the peak voltage transient.

66. The method of any one of claims 62 to 65, wherein the biphasic pulse has a pulse width of between 20 μs and 200 μs per phase.

67. The method of claim 66, wherein the biphasic pulse has a pulse width of about 25 μs per phase.

68. The method of any one of claims 65 to 67, wherein a reduction in bioimpedance of about 5% relative to a control indicates impaired barrier function in the gastrointestinal tract of a subject and/or an inflammatory disorder.

69. The method of any one of claims 65 to 67, wherein a reduction in bioimpedance of about 10% relative to a control indicates impaired barrier function in the gastrointestinal tract of a subject and/or an inflammatory disorder.

70. The method of any one of claims 53 to 69, wherein bioimpedance is determined using a method defined in any one of claims 1 to 12 or a system according to any one of claims 13 to 46.
